(19) United States
(12) Patent Application Publication
(10) Pub. No.: US 2005/0038385 A1
(43) Pub. Date: Feb. 17, 2005

(54) Retracting device for a safety syringe

(76) Inventors: Ting Shen, Shanghai (CN); Su-Ming Chang, Nantou Hsien (TW)

Correspondence Address: VARNDELL & VARNDELL, PLLC, 106-A S. COLUMBUS ST., ALEXANDRIA, VA, 22314, US

(21) Appl. No.: 10/947,369

(22) Filed: Sep. 23, 2004

Related U.S. Application Data
(63) Continuation-in-part of application No. 10/224,482, filed on Aug. 21, 2002.

(51) Int. Cl. A61M 5/00; A61M 5/32

(52) U.S. Cl. 604/110; 604/198

(57) ABSTRACT

A safety syringe has a barrel, a needle hub movably received in the barrel and having an annular shoulder formed on an inner periphery of the needle hub and a needle extending out from the needle hub. When a hook engages with the annular shoulder and the plunger is moving forward, the stop and the needle hub are free of engagement with one another and thus the needle is slanted relative to the plunger for destruction of the needle.

### CROSS REFERENCE

This application is a continuation-in-part (CIP) of co-pending application of Ser. No. 10/224,482 filed by the same applicant of this application on Aug. 21, 2002. The content thereof is hereinafter incorporated for reference.

### BACKGROUND OF THE INVENTION

1. Field of the Invention

The present invention relates to a retracting device for a safety syringe, and more particularly to a retracting device which is able to satisfactorily pull the needle hub back into the barrel so that the syringe is able to be safely disposed.

2. Description of Related Art

With reference to FIG. 8, a conventional safety syringe is shown to have a barrel (5), a needle hub (6) and a plunger (7).

The barrel (5) has a step (51) formed on an inner face thereof. The needle hub (6) has a flange (61) corresponding to the step (51) and having a diameter slightly larger than a diameter of the step (51). The plunger (7) has a stop (71) and a hook (72) formed on a distal end of the plunger (7). The needle hub (6) further defines an annular cutout (62) to receive therein an engaging portion (711) of the stop (71) and a retaining portion (63) formed to maintain the stop (71) to be in engagement with the needle hub (6). The stop (71) has an extension (712) extending out of the annular cutout (62) to correspond to the hook (72).

When the needle hub (6) is received in the barrel (5), because the diameter of the flange (61) is larger than the step (51), the diameter of the step (51) is enlarged so that the engagement between the needle hub (6) and the barrel (5) is secured. Then, the engaging portion (711) of the stop (71) is received in the annular cutout (62) of the needle hub (6).

When the plunger (7) is pushed in the barrel (5) toward the needle hub (6), the hook (72) first passes over the extension (712) of the stop (71) so as to complete an injection. Thereafter, when the user is pulling the needle hub (6) together with the needle (not shown) back into the barrel (5) in order to safely dispose of the syringe, the hook (72) engages with the extension (712) of the stop (71) and then the user is able to pull the needle hub (6) together with the stop (71) inward to the inside of the barrel (5).

However, problems often happen to the users when trying to dispose of the syringe of this type. That is, if the engagement between the flange (61) and the step (5 1) is too tight, the user will have difficulty pulling the needle hub (6) back into the barrel (5) and if the engagement between the flange (61) and the step (5 1) is too loose, the needle hub (6) will automatically fall into the barrel (5) when the user is trying to fill the syringe. Accordingly, a precise calculation of the dimension of the needle hub (6), the plunger (7) and even the barrel (5) should be taken carefully into consideration so that a proper engagement among the needle hub (6), the plunger (7) and the barrel (5) is available, which requires costly precision while manufacturing the syringe. Furthermore, when the hook (72) first passes over the extension (712), due to the rigidity of the hook (72) and the extension (712), the user will have to apply a greater force to extend the hook (72) over the extension (712). Under such a situation, the patient might feel very uncomfortable.

A conventional syringe is shown in FIG. 9, and has a plunger (10) with a first end (12) integrally provided with an assembly seat (14) to be assembled with a stop (11).

With reference to FIGS. 10 and 11, it is noted that the assembly seat (14) has a centrally defined recess (16) to receive therein a hook (18) and glue (19). That is, after the stop (11) is mounted on the assembly seat (14), the glue (19) is poured inside the recess (16) and the hook (18) is inserted into the recess (16) to have a portion of the hook (18) immersed in the glue (19). After the glue (19) has hardened, the hook (18) is fixedly received in the recess (16).

However, because the hook (18) is normally made of metal and metal is not compatible with the glue (19) inside the recess (16) such that when the hook (18) is employed to retract the needle hub (not shown), the hook (18) may come out of the recess (16) and thus the needle hub is not able to be retracted successfully.

Furthermore, with reference to FIGS. 12 and 13, it is noted that the conventional hook (18) is so configured that after the hook (18) engages with the flange inside the needle hub (6), a bottom face of the needle hub engages with a top face of the stop (11). Therefore, the engagement between the bottom face of the needle hub and the stop (11) is sustained inside the barrel and thus the user is not able to have the needle slanted inside the barrel so as to destroy the needle.

To overcome the shortcomings, the present invention tends to provide an improved retracting device for a safety syringe to mitigate and obviate the aforementioned problems.

### SUMMARY OF THE INVENTION

The primary objective of the present invention is to provide an improved retracting device for a safety syringe so that the user is able to readily pull the needle hub back into the barrel and incline the needle hub inside the barrel for destruction.

In order to accomplish the foregoing objective, the syringe has a barrel, a plunger movably received in the barrel and provided with a hook integrally formed with a stop which is formed on a distal end of the plunger, a needle hub movably received in the barrel and having a flange formed on an inner periphery of the needle hub to correspond to the hook. Engagement between the needle hub and the hook enables the needle together with the needle hub to be readily retracted into the barrel so that the needle and the needle hub are able to be slanted relative to the barrel after the needle hub and the needle are retracted into the barrel. Thus the user is able to destroy the needle inside the barrel by pushing the needle hub together with the needle to abut an inner periphery of the barrel. Therefore accidental pricking of the paramedic personnel is prevented.

Other objects, advantages and novel features of the invention will become more apparent from the following detailed description when taken in conjunction with the accompanying drawings.

### BRIEF DESCRIPTION OF THE DRAWINGS

FIG. 1 is a schematic cross sectional view showing the inner structure of the present invention;

FIG. 2 is a perspective view showing the structure of the plunger of the present invention;

FIG. 3 is an exploded cross sectional view showing the inner structure of the stop on top of the plunger;

FIG. 4 is a schematic cross sectional view showing the engagement between the hook and the stop due to the glue being received inside the stop;

FIG. 5 is an exploded perspective view of another embodiment of the present invention;

FIG. 6 is a schematic side plan view showing the engagement between the hook and the needle hub;

FIG. 7 is a schematic side plan view showing the result after the application of the present invention;

FIG. 8 is a cross sectional view showing the assembly of the barrel, the needle hub, the stop and the plunger of a conventional safety syringe;

FIG. 9 is an exploded perspective view of the structure of a conventional plunger;

FIG. 10 is a schematic view in partial cross section to show the inner structure of the assembly seat on top of the conventional plunger;

FIG. 11 is a schematic view in partial cross section to showing the assembly of a conventional hook in the assembly seat;

FIG. 12 is a schematic view showing the engagement between the stop and the needle hub in a conventional syringe; and

FIG. 13 is a schematic view showing the effect of the conventional retracting device in the safety syringe.

### DETAILED DESCRIPTION OF THE PREFERRED EMBODIMENT

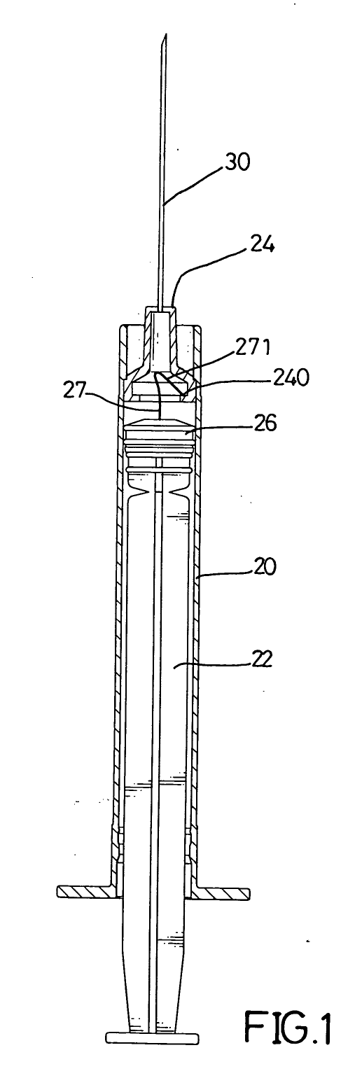

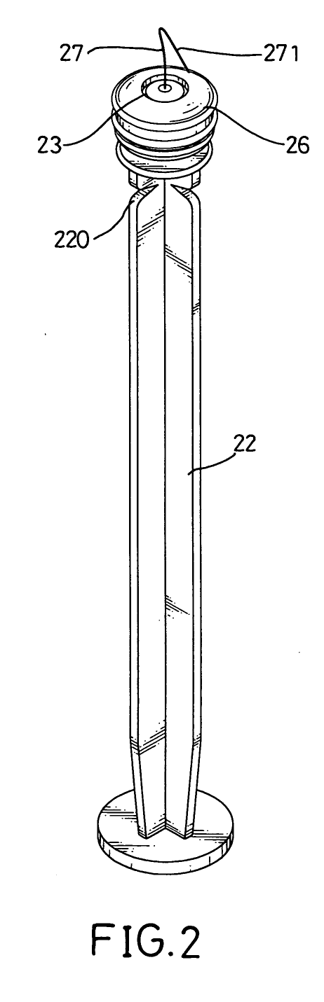

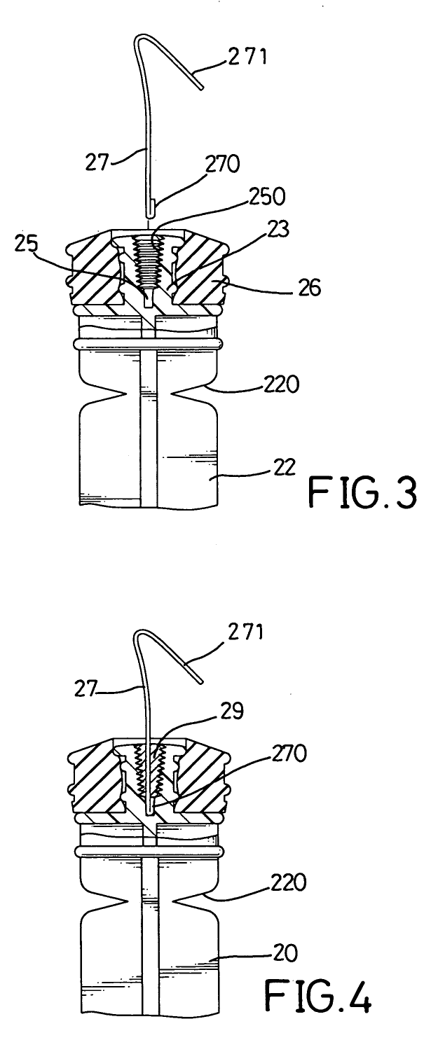

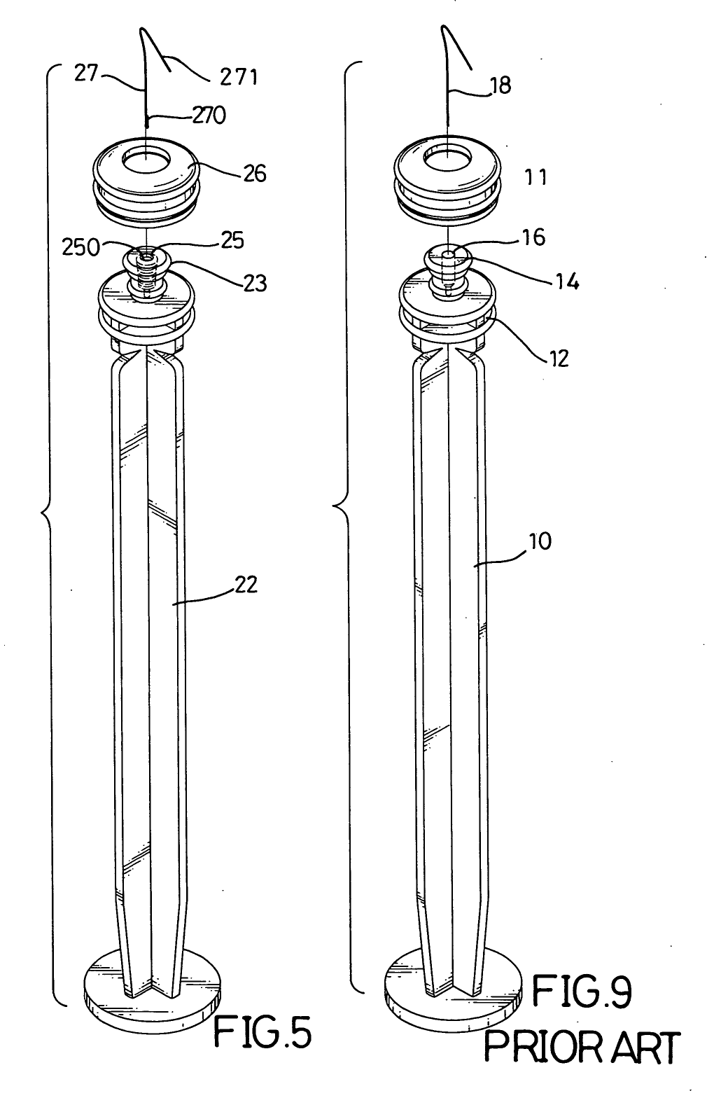

With reference to FIGS. 1 to 5, the safety syringe in accordance with the present invention has a barrel (20), a plunger (22), a stop (26) and a needle hub (24) with a needle (30) securely and fixedly extending out therefrom and an annular shoulder (240) defined in an inner periphery of the needle hub (24).

The plunger (22) is movably received in the barrel (20) and has an assembly seat (23) formed on a distal end of the plunger (22) and having a centrally defined recess (25) defined in the assembly seat (25), wherein the recess (25) has a threading (250) defined in an inner periphery defining the recess (25).

The stop (26) is to be mounted around the assembly seat (23) to function as a seal while the plunger (22) is received in the barrel (20). That is, the stop (26) engages with an inner periphery of the barrel (20) when the plunger (22) is moved inside the barrel (20).

A hook (27) is to be mounted in the recess (25) and has a head (270) formed on a first end of the hook (27) and an engaging end (271) formed on a second end of the hook (27) to correspond to the annular shoulder (240) of the needle hub (24). An adhesive element (29) is to be inserted into the recess (25). Preferably, the adhesive element (29) is glue.

Therefore, when the adhesive element (29) is received and hardens in the recess (25), the locking force of the inner periphery defining the recess (25) to the adhesive element (29) is enhanced due to the formation of the threading (250). Thus after the head (270) is immersed in the adhesive element (29), the engagement of the adhesive element (29) to the head (270) of the hook (27) is secured.

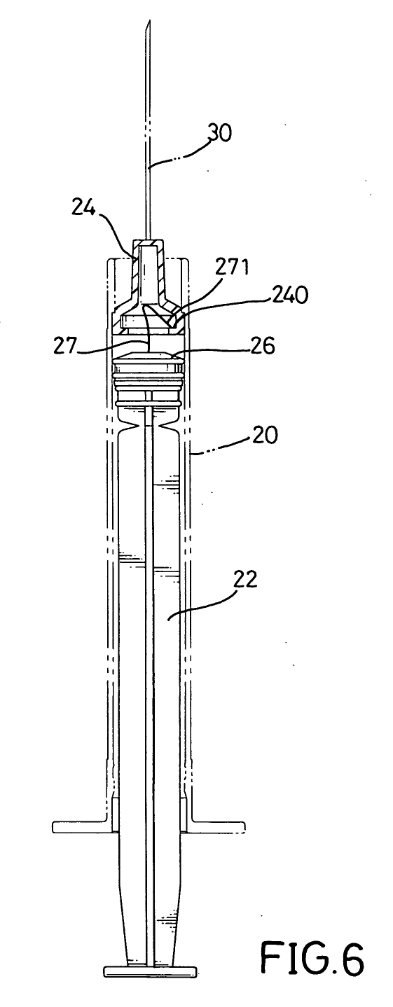

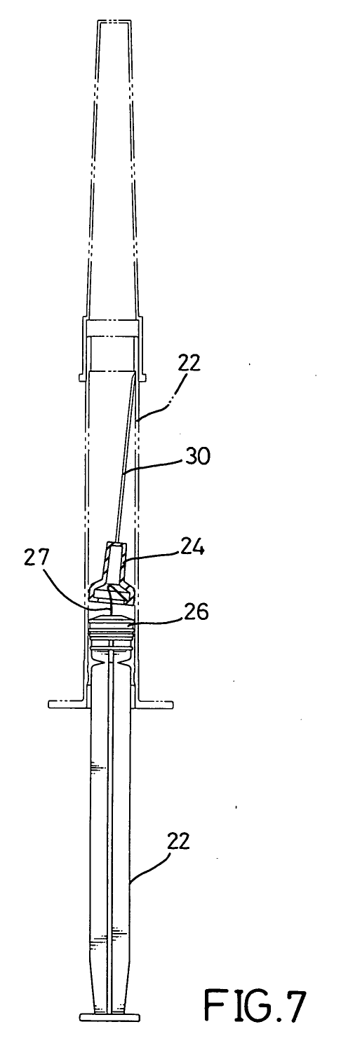

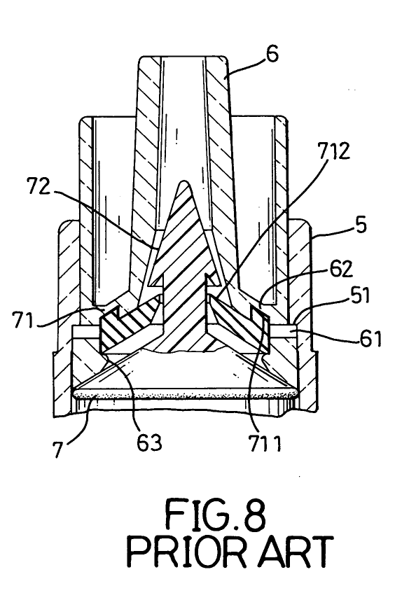

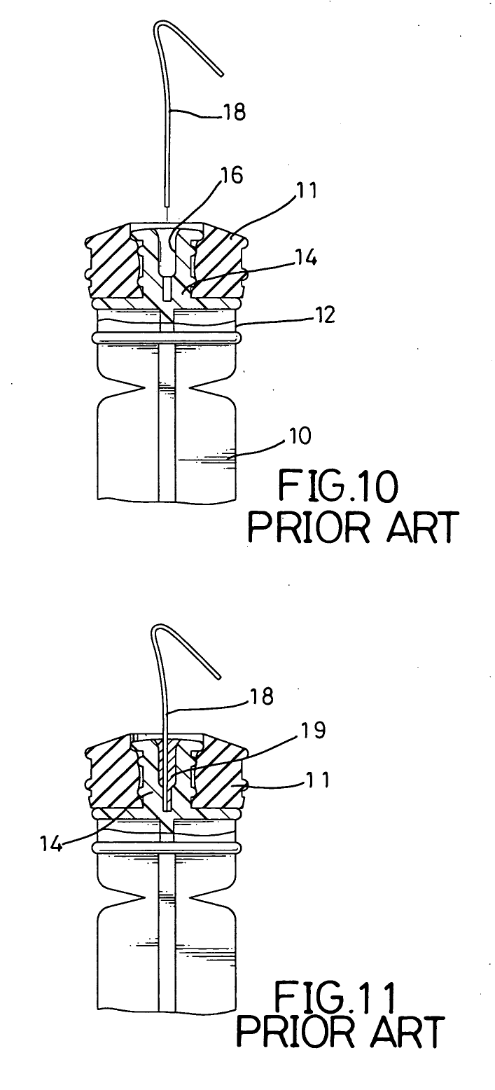

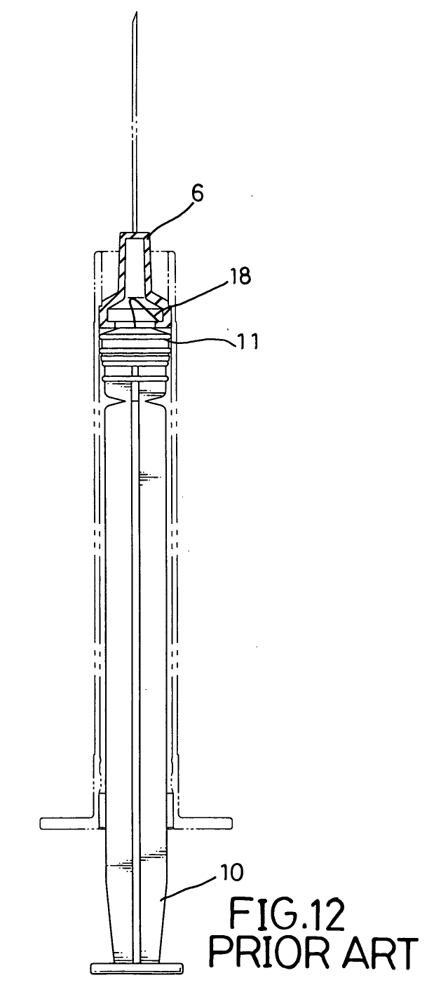

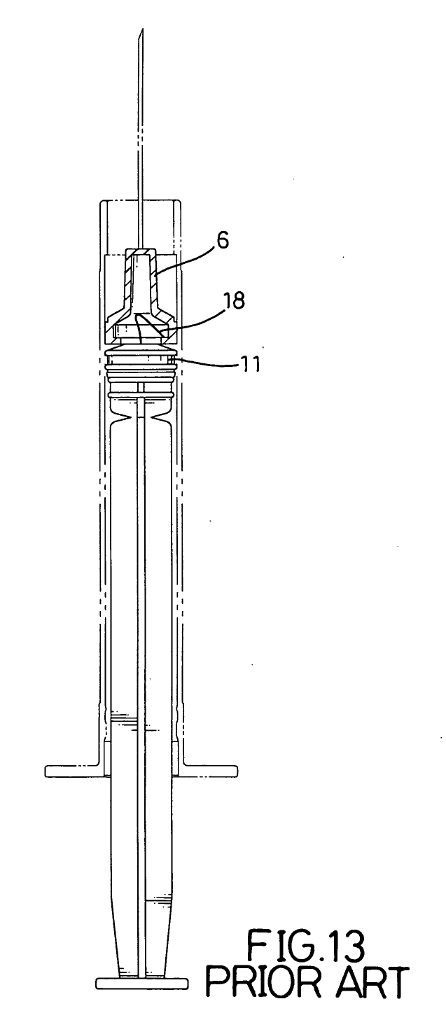

With reference to FIGS. 6 and 7, it is noted that after the plunger (22) is pushed forward inside the barrel (20), the hook (27) is extended into the needle hub (24) to have the engaging end (270) engaged with the annular shoulder (240). In particular, the hook (27) is so configured that after the engaging end (271) engages with the annular shoulder (240) of the needle hub (24), a portion of the hook (27) is between a bottom face of the needle hub (24) and a top face of the stop (26) to avoid engagement between the needle hub (24) and the stop (26). Thus after the needle hub (24) together with the needle (30) is pulled back in the barrel (20) by the plunger (22), forward movement of the plunger (22) again in the barrel (22) allows the needle hub (24) and the needle (30) to be inclined relative to the plunger (22) such that the user is able to destroy the needle (30) by abutment of the needle (30) to an inner periphery of the barrel (20).

That is, the hook (27) has a length to allow free of engagement between the needle hub (24) and the stop (26) when the needle hub (24) and the needle (30) are pulled back into the barrel (20). Therefore, due to the space between the needle hub (24) and the stop (26), the needle hub (24) together with the needle (30) is easily slanted relative to the plunger (22) and thus the needle (30) is easy to be destroyed.

It is to be understood, however, that even though numerous characteristics and advantages of the present invention have been set forth in the foregoing description, together with details of the structure and function of the invention, the disclosure is illustrative only, and changes may be made in detail, especially in matters of shape, size, and arrangement of parts within the principles of the invention to the full extent indicated by the broad general meaning of the terms in which the appended claims are expressed.

1. In a safety syringe having a barrel, a needle hub movably received in the barrel and having a needle securely attached to a distal end of the needle hub and a plunger slidably received in the barrel and having a stop formed on a first distal end of the plunger, wherein the improvements comprise:
the needle hub has an inner shoulder formed on an inner periphery of the needle hub; and
the plunger has a hook adapted to be integrally formed on a top face of the stop to correspond to the annular shoulder of the needle hub, wherein the hook is so configured that when an engaging end formed on a first end of the hook is adapted to engage with the annular shoulder, the needle hub and the stop are free of contact with each other to allow the needle hub to be inclined relative to the plunger due to a forward movement of the plunger such that the needle is destroyed by abutment to an inner periphery of the barrel.

2. The safety syringe as claimed in claim 1, wherein an assembly seat is mounted on a distal end of the plunger to be combined with the stop, the assembly seat has a centrally defined recess and a threading formed on an inner periphery defining the recess to receive therein an adhesive element so that when a head formed on a second end of the hook is received in the adhesive element which is received in the recess, engagement to the hook by the needle hub is enhanced due to the formation of the threading.

3. In a safety syringe having a barrel, a plunger movably received in the barrel, an assembly seat formed on a distal end of the plunger and having a stop mounted around the assembly seat, a needle hub movably received in the barrel and having a needle securely attached to a distal end of the needle hub, wherein the improvements comprise:
the plunger has a hook adapted to be extended out from the plunger to correspond to an annular shoulder of the needle hub, wherein the hook is so configured that the needle hub and the stop are free of contact with each other when an engaging end formed on a first end of the hook is adapted to engage with the annular shoulder to allow the needle hub to be inclined relative to the plunger due to a forward movement of the plunger such that the needle is destroyed by abutment to an inner periphery of the barrel.

4. The safety syringe as claimed in claim 3, wherein an assembly seat is mounted on a distal end of the plunger to be combined with the stop, the assembly seat has a centrally defined recess and a threading formed on an inner periphery defining the recess to receive therein an adhesive element so that when a head formed on a second end of the hook is received in the adhesive element which is received in the recess, engagement to the hook by the needle hub is enhanced due to the formation of the threading.

5. In a safety syringe having a barrel, a plunger movably received in the barrel, an assembly seat formed on a distal end of the plunger and having a stop mounted around the assembly seat, a needle hub movably received in the barrel and having a needle securely attached to a distal end of the needle hub, wherein the improvements comprise:
the plunger has a hook adapted to be extended out from the plunger to correspond to an annular shoulder of the needle hub, wherein the hook is so configured that
when an engaging end formed on a first end of the hook engages with an annular shoulder inside the needle hub and the plunger is moved backward inside the barrel, the needle hub together with the needle is straight in relation to the plunger,
when the plunger is moving forward inside the barrel with the engaging end securely engaged with the annular shoulder of the needle hub, the needle hub together with the needle is slanted with respect to the plunger such that the needle is able to engage with an inner periphery of the barrel and be destroyed as the plunger continues to move forward.

6. The safety syringe as claimed in claim 5, wherein an assembly seat is mounted on a distal end of the plunger to be combined with the stop, the assembly seat has a centrally defined recess and a threading formed on an inner periphery defining the recess to receive therein an adhesive element so that when a head formed on a second end of the hook is received in the adhesive element which is received in the recess, engagement to the hook by the needle hub is enhanced due to the formation of the threading.
